(19) United States
(12) Patent Application Publication
(10) Pub. No.: US 2024/0398901 A1
(43) Pub. Date: Dec. 5, 2024

(54) PROCESS FOR MANUFACTURING ORALLY DISINTEGRATING TABLET COMPRISING CYTOKINE

(71) Applicant: AINOS INC. TAIWAN BRANCH (USA), New Taipei City (TW)

(72) Inventors: Tsung-Fu YU, Taichung City (TW); Yi-Yu TIEN, Chiayi City (TW); Chun-Hsien TSAI, New Taipei City (TW); Ting-Chuan LEE, New Taipei City (TW); Chun-Jung TSAI, New Taipei City (TW)

(73) Assignee: AINOS INC. TAIWAN BRANCH (USA), New Taipei City (TW)

(21) Appl. No.: 18/733,102

(22) Filed: Jun. 4, 2024

Related U.S. Application Data
(60) Provisional application No. 63/506,342, filed on Jun. 5, 2023.

(51) Int. Cl. A61K 38/21; A61K 9/20

(52) U.S. Cl. CPC A61K 38/212; A61K 9/2013; A61K 9/2018; A61K 9/2095

(57) ABSTRACT

The present application provides a process for manufacturing an orally disintegrating tablet (ODT) comprising a cytokine as an active pharmaceutical ingredient comprising: acidifying an excipient, conducting a first granulation step of the acidified excipient to obtain acidic powders, and conducting a second granulation step by mixing the acidic powders and the cytokine to obtain granules containing the cytokine. The present application also provides an ODT manufactured by the process.

### BACKGROUND OF THE INVENTION

### 1. Field of the Invention

The present application relates to a process for manufacturing an orally disintegrating tablet (ODT), and more particularly to the process for manufacturing an ODT comprising cytokine.

### 2. Description of the Related Art

Cytokines are small proteins (approximately 6 to 70 kDa) important in cell signaling. Due to their size, cytokines cannot cross the lipid bilayer of cells to enter the cytoplasm and therefore typically exert their functions by interacting with specific cytokine receptors on the target cell surface. Cytokines have been shown to be involved in autocrine, paracrine and endocrine signaling as immunomodulating agents.

Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Some cytokines enhance or inhibit the action of other cytokines in complex ways. Cytokines are important in health and disease, specifically in host immune responses to infection, inflammation, trauma, sepsis, cancer, and reproduction.

Interferons (IFNs) are proteins made by host cells in response to the presence of pathogens such as viruses, bacteria, parasites, or tumor cells. IFNs allow for communication between cells to trigger the protective defenses of the immune system that eradicate pathogens or tumors. Of the 9 different families of human interferon that have been identified, interferon-alpha (IFN-α) is the most widely studied. The US FDA has approved the use of IFNα in mega doses given by injection for treating several cancers as well as hepatitis B and C. High dosage of interferon, for example, more than 1 million IU is conventionally administered by intramuscular injection to systemically treat symptoms.

Recently, oral administration of interferon has been shown in published studies to activate dozens of immune system genes in the peripheral blood and be effective against viral and autoimmune diseases, without the side effects associated with high-dose injections. However, the protein stability of the orally administered interferon is still a challenge.

The oral administration can be via such as ODT, which is able to rapidly disintegrates with saliva or a small amount of water. Drug compliance can be enhanced by application of ODT because it is convenient and easy administration for patients. However, ODT is a newly developed dosage form and some technical problems such as tablet properties still exist.

As such, an ODT containing cytokine such as interferon with good protein stability is still needed.

### SUMMARY

The present application provides a process for manufacturing an ODT comprising a cytokine as an active pharmaceutical ingredient comprising a cytokine as an active pharmaceutical ingredient comprising: acidifying an excipient, conducting a first granulation step of the acidified excipient to obtain acidic powders, and conducting a second granulation step by mixing the acidic powders and the cytokine to obtain granules containing the cytokine.

The present application also provides a process for manufacturing an ODT comprising a cytokine as an active pharmaceutical ingredient comprising: acidifying a first excipient, conducting a first granulation step of the acidified first excipient to obtain first acidic powders, sieving and granulating the first acidic powders to obtain second acidic powders, conducting a second granulation step by mixing the second acidic powders and the cytokine to obtain first granules.

### BRIEF DESCRIPTION OF THE DRAWINGS

FIG. 1 shows, in accordance with one embodiment of the present application, a process for manufacturing an ODT comprising a cytokine as an active pharmaceutical ingredient.

FIG. 2 shows the protective effect of various factors on IFN-α.

FIG. 3 shows the test results for the effect of the excipient acidification step of the process of the present application, in which IFN-α content measured using ELISA at OD450, with five samples in each group, labeled as acidification (S1-S5) and non-acidification (S1-S5).

FIG. 4 shows the test results for the effect of the processing step for the first acidic powders.

FIG. 5 shows the test results for the effect of the equipment used in the second granulation step.

FIG. 6 shows the test results for the effect of tablet thickness on IFN-α potency.

### DETAILED DESCRIPTION OF THE EMBODIMENTS

The present application provides a process for manufacturing an ODT comprising a cytokine as an active pharmaceutical ingredient.

The cytokine is selected from a group consisting of chemokines, interferons, interleukins, lymphokines, and tumor necrosis factors. The cytokine can be a native cytokine, a recombinant cytokine, or a mixture thereof. The dosage of the cytokine contained in the ODT is not particularly limited, which can be decided depending on situation.

In some embodiments, the cytokine is selected from interferon, for example, type I INFs, type II INFs, and type III INFs. In one embodiment, the cytokine is interferon-alpha (INF-α), more preferably, a recombinant INF-α.

In one embodiment, the ODT can contain a dosage of 1-100,000 International Units (IU) of INF-α. In one embodiment, the ODT can contain a dosage of equal to or less than 1,000 microgram of INF-α. The dosage can be, for example, less than 75,000 IU, less than 50,000 IU, less than 25,000 IU, less than 10,000 IU, less than 7,500 IU, less than 5,000 IU, less than 2,500 IU, less than 1,000 IU, more than 10 IU, more than 100 IU, more than 250 IU, more than 500 IU, more than 750 IU, more than 900 IU, or a value between any two of the above numerical points.

In the present application, the ODT comprises at least one excipient. The excipient is a pharmaceutically acceptable ingredient and known excipients may be used as long as it can function as an excipient in ODT and does not adversely affect the cytokine. In embodiments, the excipient can include, for example, sugars, sugar alcohols, cellulose, polysaccharides, stearic acid, magnesium stearate, calcium stearate, polyethylene glycol, sodium lauryl sulfate, glycerin, diclacium phosphate anhydrous, magnesium metasilicate aluminate, and silicon oxides. Examples of sugars include lactose, maltose, dextrin, trehalose, sucrose and the like. Examples of sugar alcohols include mannitol, erythritol, isomalt, lactitol, maltitol, sorbitol, xylitol and the like. Examples of cellulose include microcrystalline cellulose, silicified microcrystalline cellulose, powdered cellulose and the like. Examples of polysaccharides include starch such as corn starch, potato starch, pregelatinized starch and the like. In some embodiments, the excipient is sugar. In a preferred embodiment, the excipient is maltose.

The process for manufacturing an ODT comprising a cytokine as an active pharmaceutical ingredient comprises the following step: acidifying an excipient, conducting a first granulation step of the acidified excipient to obtain acidic powders, and conducting a second granulation step by mixing the acidic powders and the cytokine to obtain granules containing the cytokine.

In the present application, the excipient is acidified before mixing with the active pharmaceutical ingredient, i.e. the cytokine. The acidified excipient is able to prevent from protein degradation of the cytokine during the manufacture of ODT, and maintain the stability and the content of the cytokine in the final product.

In some embodiments, the acidified excipient is prepared by the following steps: mixing an acid and water to obtain an acid solution, and mixing the excipient and the acid solution. The water can be purified water, sterile water, deionized water, and the like.

The acid is added to adjust the pH of the excipient solution. In some embodiments, the acidification makes the solution of the excipient have a pH of 3 to 10, for example, the pH of 3.5 to 8, the pH of 4 to 7, or the pH of 5 to 6. An inorganic acid and/or an organic acid can be used as long as it does not destroy or change the components or properties of the ODT. Examples of the acid can include, without limitation, hydrochloric acid, sulfuric acid, nitric acid, phosphoric acid, carbonic acid, acetic acid, citric acid, malic acid, succinic acid, fumaric acid, lactic acid and the like. In some embodiments, the acid is citric acid.

For the granulation steps in the present application, there is no particular limitation and conventional granulation methods may be applied. Examples of the granulation methods include high-speed shear mixing granulation, fluidized bed granulation, centrifugal granulation, roller granulation, spray granulation and the like. Considering the properties of the active pharmaceutical ingredient contained in the ODT, wet granulation methods are preferred. In some embodiments, high-speed shear mixing granulation is applied. In some embodiments, fluidized bed granulation is applied.

After the excipient acidification step and the first granulation step, the obtained acidic powders are further processed. Hereafter the acidic powders obtained from the first granulation step are sometimes called “first acidic powders”. The first acidic powders can be sieved by, for example, using a 100-mesh screen, in which the powders passing the screen (called “the sieved part of the first acidic powders” or “undersize material” hereafter) and the powders being retained on the screen (“the retained part of the first acidic powders” hereafter) are separated. The sieved part of the first acidic powders are further mixed with the excipient which is not acidified, and then granulated to obtain second acidic powders.

Subsequently, the acidic powders can be mixed with the cytokine.

The cytokine can be contained in a solution. The solution can further comprises a solvent and an acid. In one embodiment, the solution comprising the cytokine, the solvent and the acid has a pH of 3 to 10, for example, the pH of 3.5 to 8, the pH of 4 to 7, or the pH of 5 to 6. The solvent can be any pharmaceutically acceptable solvent as long as it does not adversely affect the cytokine. In one embodiment, the solvent can be water, such as purified water, deionized water, and/or sterile water. The acid can be an inorganic acid and/or an organic acid as long as it does not adversely affect the cytokine. Examples of the acid can include, without limitation, hydrochloric acid, sulfuric acid, nitric acid, phosphoric acid, carbonic acid, acetic acid, citric acid, malic acid, succinic acid, fumaric acid, lactic acid and the like. In some embodiments, the acid is hydrochloric acid (HCl). In some embodiments, the solution further comprises an excipient, which can be identical to or different from the excipient subjected to acidification. In some embodiments, the solution can further comprises an additive selected from a group consisting of buffers, disintegrants, binders, colorants, lubricants, fluidizing agents, surfactants, flavoring agents, antioxidants, preservatives or any combination thereof.

In some embodiments, the first acidic powders are mixed with the cytokine to proceed the second granulation step. In some embodiments, the second acidic powders are mixed with the cytokine to proceed the second granulation step.

In some preferred embodiments, the second acidic powders are mixed with the cytokine to conduct the second granulation step to obtain first granules, then the first granules are further mixed with the retained part of the first acidic powders to conduct the third granulation step to obtain second granules or the compression step to obtain the tablets.

In some embodiments, one or more additional excipients can be added in any suitable step of the process. In some embodiments, an additive such as a buffer, a disintegrant, a binder, a colorant, a lubricant, a fluidizing agent, a surfactant, a flavoring agent, an antioxidant, a preservative, a filler or any combination thereof can be added in any suitable step of the process. Such additive is not particularly limited and can be applied or added based on the general knowledge of the relevant technical field. In one embodiment, the additive is magnesium stearate.

According to the above, the present application provides, in some embodiment, a process for manufacturing an ODT comprising a cytokine comprises the following step: acidifying a first excipient, conducting a first granulation step of the acidified first excipient to obtain first acidic powders, sieving and granulating the first acidic powders to obtain second acidic powders, and conducting a second granulation step by mixing the second acidic powders and the cytokine to obtain first granules.

The present application also provides, in some embodiment, a process for manufacturing an ODT comprising a cytokine comprises the following step: acidifying a first excipient, conducting a first granulation step of the acidified first excipient to obtain first acidic powders, sieving and granulating the first acidic powders to obtain second acidic powders, conducting a second granulation step by mixing the second acidic powders and the cytokine to obtain first granules, and compressing the first granules to form the tablets.

Further, in some embodiment, the present application also provides a process for manufacturing an ODT comprising a cytokine as an active pharmaceutical ingredient comprising: acidifying a first excipient, conducting a first granulation step of the acidified first excipient to obtain first acidic powders, sieving and granulating the first acidic powders to obtain second acidic powders, conducting a second granulation step by mixing the second acidic powders and the cytokine to obtain first granules, and mixing the first granules and the first excipient and/or an additive and compressing to form tablets. In particular, the retained part of the first acidic powders can be mixed with the first granules.

In the present application, the process may further comprise a drying step, a sieving step and a compression step. The drying step can be conducted after the first granulation step, the second granulation step and/or the third granulation step. After the drying step, a sieving step may be optionally conducted to improve the uniformity of the obtained granules. The mesh size of the screen used in the sieving step can be decided depends on the needs. After completion of all granulation steps, a compression step can be conducted to compress the granules containing the cytokine to form the tablets.

In another aspect, the present application also provides an ODT manufactured by the process described in the present application. The ODT is able to maintain good and sufficient potency and stability of the contained cytokine.

The present application provides the manufacturing process of cytokine-containing ODT with optimal process steps, being suitable for industrial scaled manufacture. The process of the present application can be applied to manufacture the ODT comprising cytokine with good protein stability and cytokine potency.

### EXAMPLES

### Example 1

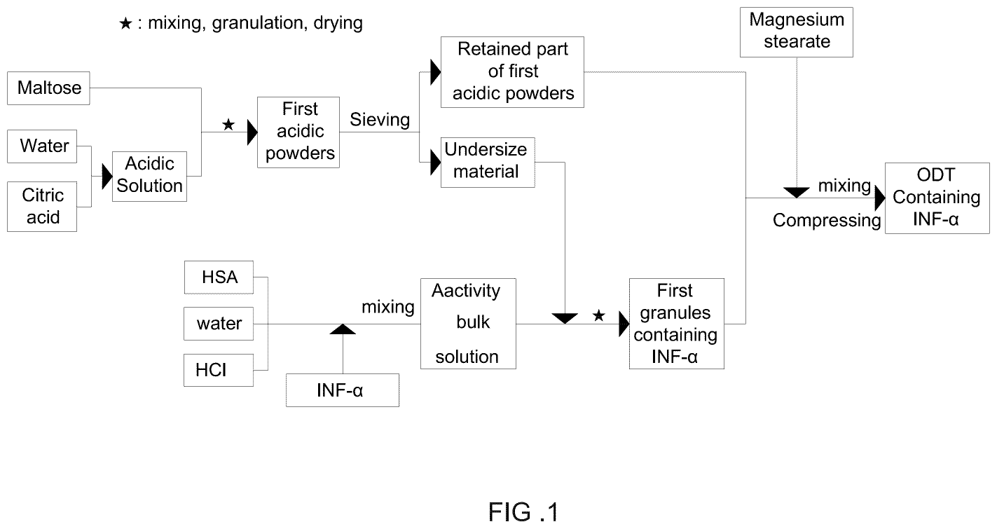

Referring FIG. 1, the steps of the process for manufacturing an ODT comprising INF-α were illustrated. In this Example, maltose was used as the excipient to be acidified.

### Preparation of Adjusted Activity Bulk Solution:

HCl for pH adjustment, sodium acetate trihydrate, and sodium chloride (NaCl) were added into purified water. Keep stirring for at least 10 mins until a clear solution by visual observation was obtained. human serum albumin was added and stirred for 10 mins. INF-α solution was slowly added into the solution, and kept stirring for 10 mins. As such, the adjusted activity bulk solution was obtained.

### Preparation of Acidic Powders:

Maltose was grinded, and passed through No. 40 mesh. The maltose powders were sieved with Comil (screen aperture 0.032 inches, 032R).

Citric acid was slowly added into purified water under stirring. Keep stirring for 10 mins to obtain an acidic solution with clear appearance.

The acidic solution and the maltose powders were mixed and fed to the high speed-mixing granulating machine to manufacture the acidic powders. The acidic powders were dried by using the fluid bed granulation dryer/coater. Optionally, the acidic powders were passed through a 30 mesh screen (Vibro-Sieve Separator) after the drying step and before the processing step. As such, the first acidic powders were obtained.

### Processing of the Acidic Powders:

The first acidic powders were sieved by using a 100 mesh screen (Vibro-Sieve Separator) to separate the undersize material (the acidic powders passing through the screen) and the retained part of first acidic powders (the acidic powders not passing through the screen).

The undersize material can be mixed with maltose powders in a ratio of 10-50 wt % of undersize material: 90-50 wt %. In this Example 1, the undersize material is 20%. The mixture was fed to the high speed-mixing granulating machine to obtain the second acidic powders.

### Preparation of Granules Containing INF-α:

The second acidic powders were mixed with the adjusted activity bulk solution. The mixture was fed to the high speed-mixing granulating machine to conduct the binder-in and granulation steps to obtain the first granules containing INF-α. Subsequently, the first granules were dried at 30° C. for 15 mins by using the fluid bed granulation dryer/coater and sieved by a 60 mesh screen (Vibro-Sieve Separator).

The first granules were mixed with the retained part of first acidic powders in the drum mixer for 10 mins. As an additional excipient, magnesium stearate was added and mixed in the drum mixer for 5 mins.

The high speed-mixing granulating machine was applied to manufacture the second granules containing INF-α.

In an alternative embodiment, after addition of magnesium stearate, the mixture could be subjected to tablet compression.

In the following examples, the different parameters of the process were changed and evaluated to find out the optimal steps/parameters/conditions for the process.

### Example 2: Evaluation of Various Factors on Protective Effect on INF-α

Experiment for finding optimal solution for adjusting actively bulk solution, this is very important for API potency. The optimal solution could preserve API potency throughout the whole manufacture process. Table 1 shows the experiment design of two factors.

TABLE 1

| | | |
|---|---|---|
| Factor | Low Level | High Level |

| | | | | |
|---|---|---|---|---|
| Sodium chloride | 31.5 | mM | 315 | mM |
| HS Albumin | 1 | mg/mL | 3 | mg/mL |

| pH 4.0 adjust with | — (pH 6.8) | pH 4.0 | |
|---|---|---|---|
| HCl | | | |
| Water for injection | Pure water | Water for injection | |
| EDTA | — | 0.5 | mg/mL |

| | | | | |
|---|---|---|---|---|
| API conc. | 3.7 × 10⁵ | IU | 1.1 × 10⁶ | IU |

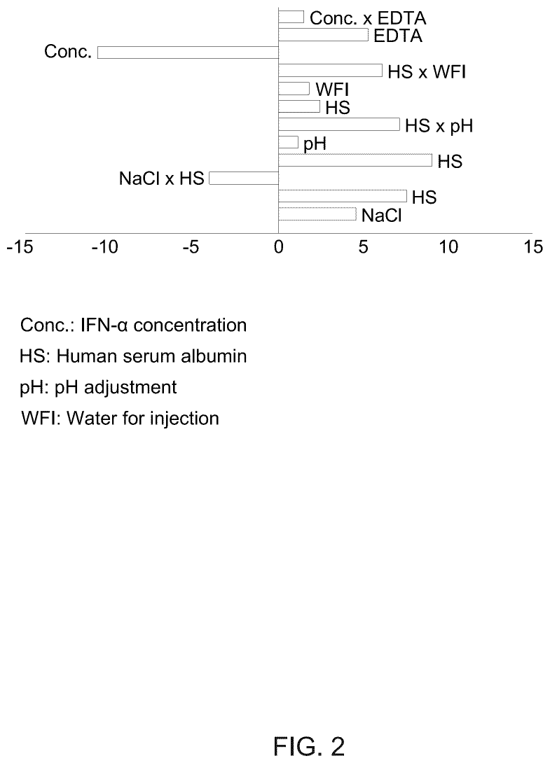

FIG. 2 shows that, for the protective effect of various factors on INF-α, the positive value has protective effect while the negative value has destructive effect on INF-α, and there is an interaction between the two factors, the positive value strengthens the protective effect of the interaction, and the negative value weakens the protective effect.

### Results

All cytokines need to stay in buffer system with surfactant/stabilizer when dissolve, but not with stressing agents. Due to most biological medicinal products are blood products, sera products, vaccines, cell therapy products, gene therapy products, biotechnology products, human or animal extracted products, allergen products, tissue engineered products. The above biological medicinal products are all sterile products. However, the ODT containing cytokine manufactured by the process of the present application is a non-sterile product. How to protect and maintain the contained INF-α potency and function in the ODT was a primary goal. The inventors have not only set up a more suitable manufacture process, but also find the optimal parameter for API. According to our study, referring FIG. 2, HS (Human Serum Albumin), NaCl, pH adjustment, WFI (Water for Injection), and EDTA have protective effects. By contrast, the higher the IFN concentration, the worse the protective effect. It can be explained that the higher the IFN concentration, the lower the HS protective agent. In addition, salts and HS are antagonistic to each other, and the protective effect becomes weaker when the concentration of the two increases, and the interaction of other experimental factors strengthens each other. Combined this finding with two manufacture processes, biological solid dosage forms will have the beneficial effect.

### Example 3: Evaluation of the Excipient Acidification Step

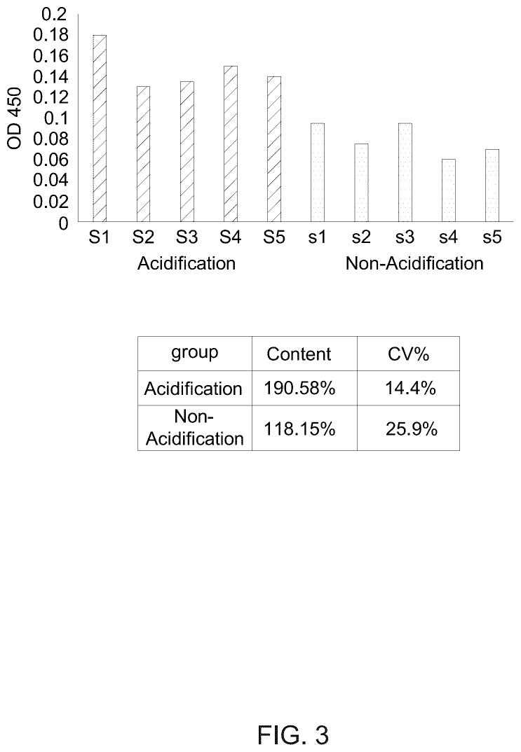

This example demonstrated the preparation method of a drug combination containing INF-α and maltose. Initially, 1 kg of maltose powder was acidified using a 0.1% citric acid solution in a High Shear Mixer (SMG), followed by granulation, and drying in a Fluid Bed Granulation Dryer (FBD) for 15 minutes to obtain acidified powder. Then, 8.9 μg of API solution, 510 mg of sodium acetate, 2.76 g of NaCl, 450 mg of HSA, and 1.65 ml of HCl were mixed with sterile or pure water to prepare 150 ml of actively bulk solution. Subsequently, 472.4 g of acidified powder and actively bulk solution were granulated and dried using an FBD. FIG. 3 showed that the INF-α content in the secondary granulated powder was measured by ELISA, and comparison between the acidified and non-acidified groups revealed higher retention in the acidified group, indicating that the excipient acidification step can effectively protect the stability of IFN-α.

### Example 4: Water Proportion in the Acidification Solution

This experiment tested the adjustment of the water proportion in the acidification solution and evaluated its impact on the granulation step. 0.5 kg of maltose powder was tested with different volumes of colored water. A more uniform particle distribution was observed when the ratio of water to maltose ranges from 6 to 8 g/100 g. However, when the water content was too low (5 g/100 g), coarse and fine particles with different shades of color were observed after granulation, indicating poor powder uniformity. On the other hand, excessive water content leaded to maltose dissolution and adherence to the walls of the container, forming large crystal aggregates that adversely affected the yield.

### Example 5: Acidification Granulation Equipment

This experiment utilized 0.5 kg of maltose powder and a 0.1% citric acid solution, and the tested equipment for granulation included the SMG and the FBD. After granulation, the particle size of the powder was examined. It was observed that the FBD group exhibited a more distinct difference in particle size, indicating a significant proportion of aggregation. In contrast, the SMG group showed a more uniform particle size distribution. Accordingly, SMG was preferred for granulation in powder form.

On the other hand, FBD was preferred for the drying step, with the temperature set at 55-60° C. and a drying time of 10-20 minutes. Water content analysis indicated approximately 1±0.5%. Similar results could be obtained using a vacuum pump, but it would require hours, whereas choosing FBD could reduce time costs and the probability of unexpected occurrences during the production process.

### Example 6: Processing of the First Acidic Powders

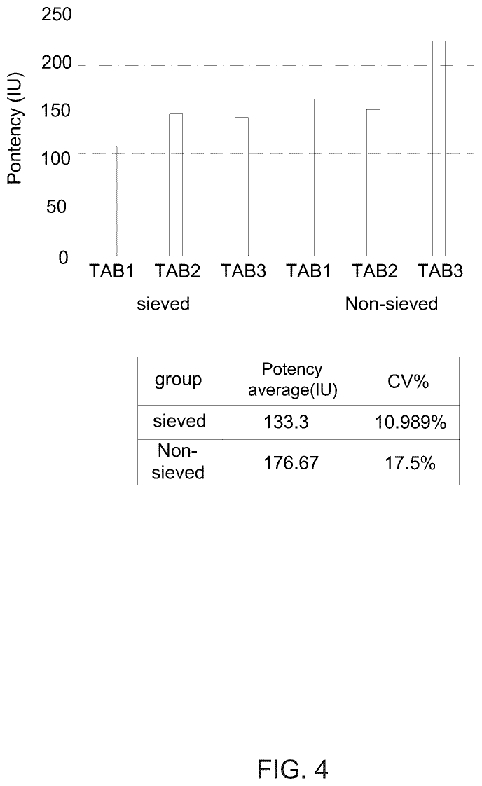

In this experiment, 1 kg of maltose powder and a 0.1% citric acid solution (61 g) were subjected to acidification granulation using a SMG and then dried for 15 minutes using a FBD. The acidified powder was sieved through a 100 mesh sieve, and 500 g of the undersized particles were mixed with the actively bulk solution and granulated/dried using FBD to obtain secondary granulated powders. Finally, the secondary granulated powder: the acidified powder:magnesium stearate with a weight ratio of 1:0.98-1:0-0.02 were mixed and tableted. The tablets were tested for INF-α potency and the results were shown in FIG. 4. For the INF-α-containing ODT, the INF-α is requested to evenly distribute in each tablet. The content of INF-α cannot be directly reflected by the test result of potency. As shown in FIG. 4, the sieved group was with variations in samples being less than 15%, indicating that the addition of a sieving step before secondary granulation improves tablet uniformity.

### Example 7: Comparison of Secondary Granulation Equipment

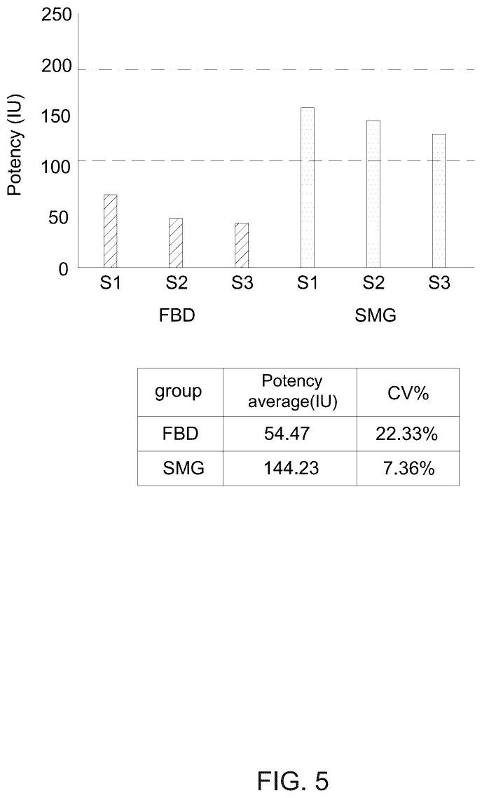

In this experiment, the tested equipment for secondary granulation included SMG and FBD. For FBD, the tablet was prepared as Example 6. For SMG, the tablet was prepared as Example 6 except the undersized material and active bulk solution were granulated using SMG and then dried using FBD to obtain the second acidic powders. The results were shown in FIG. 5, in which the variation between samples was less than 20%.

As shown in FIG. 5, SMG equipment granulation retained a higher proportion of IFN attached to the acidic powders and higher tablet uniformity than FBD.

### Example 8: Tablet Thickness

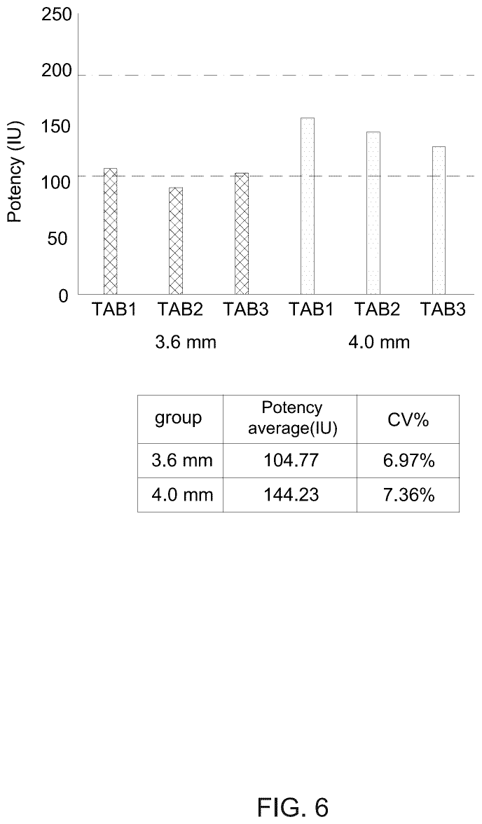

In this example, the impacts of the table thickness were evaluated. Compression parameters were adjusted to increase thickness from 3.6 mm to 4.0 mm, and the samples were tested for INF-α potency. The results were shown in FIG. 6. The data indicated that the increased thickness of the tablet made no change in uniformity, but better protection of INF-α potency.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A process for manufacturing an orally disintegrating tablet comprising a cytokine as an active pharmaceutical ingredient comprising:
acidifying an excipient,
conducting a first granulation step of the acidified excipient to obtain acidic powders, and
conducting a second granulation step by mixing the acidic powders and the cytokine to obtain granules containing the cytokine.

2. The process of claim 1, further comprising a drying step, which is conducted after the first granulation step and/or after the second granulation step.

3. The process of claim 2, further comprising a sieving step, which is conducted after the drying step.

4. The process of claim 1, further comprising: compressing the granules containing the cytokine to form tablets.

5. The process of claim 1, wherein the cytokine is selected from a group consisting of chemokines, interferons, interleukins, lymphokines, and tumor necrosis factors.

6. The process of claim 1, wherein the excipient is selected from a group consisting of sugars, sugar alcohols, cellulose, polysaccharides, stearic acid, magnesium stearate, calcium stearate, polyethylene glycol, sodium lauryl sulfate, glycerin, diclacium phosphate anhydrous, magnesium metasilicate aluminate, and silicon oxides.

7. The process of claim 1, wherein the excipient is acidified by the following steps:
preparing an acid solution comprising an acid and water, and
mixing the excipient and the acid solution,
wherein the obtained solution comprising the excipient, the acid and water has a pH of 3 to 10.

8. The process of claim 7, wherein the acid is selected from a group consisting of inorganic acids or organic acids.

9. The process of claim 1, wherein the cytokine is contained in a solution comprising a solvent and an additive.

10. The process of claim 9, wherein the solution comprising the cytokine, the solvent and the additive has a pH of 3 to 10.

11. A process for manufacturing an orally disintegrating tablet comprising a cytokine as an active pharmaceutical ingredient comprising:
acidifying a first excipient,
conducting a first granulation step of the acidified first excipient to obtain first acidic powders,
sieving and granulating the first acidic powders to obtain second acidic powders,
conducting a second granulation step by mixing the second acidic powders and the cytokine to obtain first granules.

12. The process of claim 11, further comprising a drying step and a sieving step wherein the drying step is conducted after the first granulation step, the second granulation step, and/or obtaining the second acidic powders, and the sieving step is conducted after the drying step.

13. The process of claim 11, further comprising:
compressing the first granules to form tablets.

14. The process of claim 11, further comprising:
mixing the first granules and the first excipient and/or an additive, and compressing to form tablets.

15. The process of claim 18, wherein the additive is magnesium stearate.

16. The process of claim 11, wherein the first excipient is acidified by the following steps:
preparing an acid solution comprising an acid and water, and
mixing the excipient and the acid solution,
wherein the obtained solution comprising the excipient, the acid and water has a pH of 3 to 10.

17. The process of claim 11, wherein the cytokine is contained in a solution comprising a solvent and an additive, and the solution comprising the cytokine, the solvent and the additive has a pH of 3 to 10.

18. The process of claim 17, wherein the solution comprising the cytokine, the solvent and the acid further comprises a second excipient.

19. The process of claim 11, wherein the cytokine is selected from a group consisting of chemokines, interferons, interleukins, lymphokines, and tumor necrosis factors, and the first, second or third excipient is selected from a group consisting of sugars, sugar alcohols, cellulose, polysaccharides, stearic acid, magnesium stearate, calcium stearate, polyethylene glycol, sodium lauryl sulfate, glycerin, diclacium phosphate anhydrous, magnesium metasilicate aluminate, and silicon oxides.

20. An orally disintegrating tablet (ODT) comprising a cytokine as an active pharmaceutical ingredient characterized by that: the ODT is manufactured by the process of claim 1.
